(19) United States
(12) Patent Application Publication
(10) Pub. No.: US 2005/0283124 A1
(43) Pub. Date: Dec. 22, 2005

(54) Needle with slotted tip

(76) Inventor: Timothy Simon, Los Alamitos, CA (US)

Correspondence Address: FULWIDER PATTON LEE & UTECHT, LLP, 200 OCEANGATE, SUITE 1550, LONG BEACH, CA, 90802, US

(21) Appl. No.: 11/152,921

(22) Filed: Jun. 15, 2005

Related U.S. Application Data
(63) Continuation-in-part of application No. 10/163,741, filed on Jun. 5, 2002, now abandoned.

(51) Int. Cl. A61M 5/32

(52) U.S. Cl. 604/272

(57) ABSTRACT

A manual surgical instrument needle for the injection of fluid into intraarticular space having a helical slot formed on its lateral surface and a closed off distal end. The distal end is preferably configured with a hollow bevel. The slot ensures that a direct flow path into the intraarticular space is established despite the embedment of the distal tip of the needle into articular cartilage or bone or soft tissue and regardless of the needle's rotational orientation. The hollow bevel of tip reduces friction and drag during tissue penetration to minimize trauma to the patient.

### CROSS-REFERENCES TO RELATED APPLICATIONS

This application is a continuation-in-part of U.S. patent application Ser. No. 10/163,741 filed Jun. 5, 2002 which is hereby incorporated by reference in its entirety.

### BACKGROUND OF THE INVENTION

The present invention relates to a class of manual surgical instruments which include disposable or reusable aspiration and injection needles. For simplicity, the device of the present invention will be referred to as “needle” hereafter. The present invention relates to needles used for aspiration or injection of fluids and is more particularly directed to needles used for injecting fluid into an intraarticular space.

Osteoarthritis is one of the most common and costly chronic medical conditions. At present, most therapies are directed towards minimizing pain and swelling, maintaining joint mobility and reducing associated disability. Non steroidal anti inflammatory drugs are the most widely used medications and have been the mainstay of treatment by physicians and over the counter use by patients. Alternative therapies are however gaining in popularity.

In osteoarthritis, there is often a reduction in the elastoviscosity of the synovial joint fluid secondary to a decrease in the molecular weight and concentration of hyaluronic acid. Viscosupplementation is a therapeutic technique that addresses the decrease in synovial viscosity with the injection of high molecular weight hyaluronan molecules. Viscosupplementation was initially used to treat post traumatic osteoarthritis in race horses, and later used for human knee arthritis in the early 1970's. Several human clinical trials have shown a single course of three weekly injections of hyaluronan was more effective than saline controls, and equivalent to or better than continuous non steroidal anti inflammatory drug therapy plus arthrocentesis.

Hyaluronan has been approved as an intraarticular device to coat the articular surfaces and synovial lining in the knee joint. However, in order to achieve maximal therapeutic benefit from hyaluronic acid derivative injections, the material must be delivered directly into the knee joint space as its high viscosity precludes its diffusion there into from the surrounding tissue. This is in contrast to intraarticular injections of for example cortisone wherein accurate placement is not as critical as its low viscosity allows it to readily diffuse and thereby achieve a clinical response.

Achieving accurate positioning of the distal end of an injection needle is difficult and studies have shown that clinicians are often unable to achieve proper intraarticular placement. Often, a clinician can only rely on effusion that may be present in the intraarticular space in order to confirm proper placement, whereby the ability to aspirate such fluid from the joint indicates that the needle tip is in fact positioned in the intrarticular space rather than proximally thereto in the fat pad or distally thereto, embedded in the cartilage or bone. The absence of effusion in the intraarticular space would of course preclude the use of such technique altogether and further compounds the problem as the intraarticular space is as a result much smaller. The injection of hyaluroran is therefore often less effective than it could be by virtue of the fact that it is simply not delivered to the appropriate place.

An additional problem associated with the use of the conventional hypodermic needles to gain access to the intraarticular space is that the hollow configuration of the distal end of the needle has a cookie cutter effect and therefore has a tendency to detach a plug of synovial tissue which is then injected into the joint along with the hyaluronan. The presence of such debris within the intraarticular space has a deleterious effect and is therefore to be avoided.

An improved device is therefore needed with which the intraarticular space can readily be accessed with minimal effort and without the need to rely on effusion to confirm proper placement. Additionally, it is highly desirable to be able to access the intraarticular space without the risk of transferring detached particles of synovial tissue there into.

### SUMMARY OF THE INVENTION

The needle of the present invention overcomes the shortcomings of devices previously used for injecting fluids into intraarticular spaces. The use of the needle greatly simplifies the clinician's task and ensures that injectant reaches the intraarticular space without requiring insertion to a precise depth, precisely maintaining such depth and without the need to rely on the presence of effusion to confirm placement. Additionally, the needle of the present invention prevents the detachment of synovial tissue and the subsequent transfer of such tissue into the intraarticular space. Finally, the device of the present invention is preferably configured to maximize strength and to enhance its tissue penetrating ability without compromising its tracking ability in order to allow the clinician to accurately control the path of the needle as it is advanced through tissue.

The needle of the present invention has a helical slot formed in its lateral surface. The slot extends from just proximal to the needle's tip along approximately 9.0 10.0 mm of its longitudinal length and subtends an angle of about 360°. The helical nature of the slot ensures that the interior of the needle is set into direct fluid communication with intraarticular space upon embedment of the needle tip in the articular cartilage and irrespective of its rotational orientation.

The needle tip is sealed off in order to prevent the detachment and capture of tissue as the needle is advanced through the various layers of tissue. The needle is preferably configured with a hollow bevel tip in combination with a recessed plug that is disposed immediately proximal to the bevel. The hollow bevel serves to reduce the force needed for penetrating tissue as the total surface area that engages skin tissue is thereby substantially reduced while the plug prevents the entry of tissue into the lumen of the needle. More preferably, needle is constructed of two concentric tubes wherein the outer tube has the bevel formed therein while the inner tube has a closed off distal end which serves as the recessed plug for the outer tube. The dual tube configuration not only serves to enhance needle strength but additionally simplifies the manufacture and fitment of a recessed plug to a hollow needle. A temporarily inserted trochar may be used to keep tissue from entering the lumen of the needle and to enhance the strength of the needle during the insertion step.

These and other features and advantages of the present invention will become apparent from the following detailed description of a preferred embodiment which, taken in conjunction with the accompanying drawings, illustrates by way of example the principles of the invention.

### BRIEF DESCRIPTION OF THE DRAWINGS

FIGS. 1A-C are side views of the needle of the present invention in successive rotational orientations;

FIG. 2 is a side view of an alternative embodiment of the needle of the present invention;

FIG. 3 is a side view of a further alternative embodiment of the needle of the present invention;

FIG. 4 is a side plan view of the needle shown in FIG. 3;

FIGS. 5A-D are side plan views of plugs for use in the needle shown in FIGS. 3 and 4;

FIGS. 6A-E illustrate a method for manufacturing a needle as is shown in FIGS. 3 and 4, wherein FIG. 6D is enlarged view of the circled section shown in FIG. 6C;

FIGS. 7A and 7B is are side views of another preferred alternative embodiment of the present invention;

FIG. 8 is a side view of a trochar for use during the placement of the needle of the present invention; and

FIG. 9 is an enlarged cross sectional view of a knee joint with the needle of the present invention inserted thereinto.

### DETAILED DESCRIPTION OF THE INVENTION

The needle of the present invention facilitates the injection of fluid into the intraarticular space. The device ensures the flow of fluid directly into the space despite the embedment of the needle tip in cartilage and/or despite only minimal spacing between the cartilage and the fat pad. Moreover, the reliance on effusion is not necessary for confirming the proper placement of the needle.

FIGS. 1A-C illustrate the needle 12 of the present invention which has a helical slot 14 formed in its lateral surface. The distal end 16 of the slot is located just proximal to the tapered portion of the needle's distal tip 18 while the proximal end 20 of the slot is distanced approximately 0.3 mm from its distal end 22 as measured along the needle's longitudinal axis 24. The slot spirals along the needle's lateral surface so as to subtend an angle of approximately 360°. The slot preferably has a constant width along its entire length of approximately 10.3 mm and has rounded ends 16, 20.

In the embodiment illustrated in FIGS. 1A-C, the needle tip 18 is closed off and has a conical form wherein the distal end 22 of the tip is aligned with the needle's longitudinal axis 24. The outer surface of the conical tip preferably defines an angle 26 of approximately 12° relative to the needle's longitudinal axis.

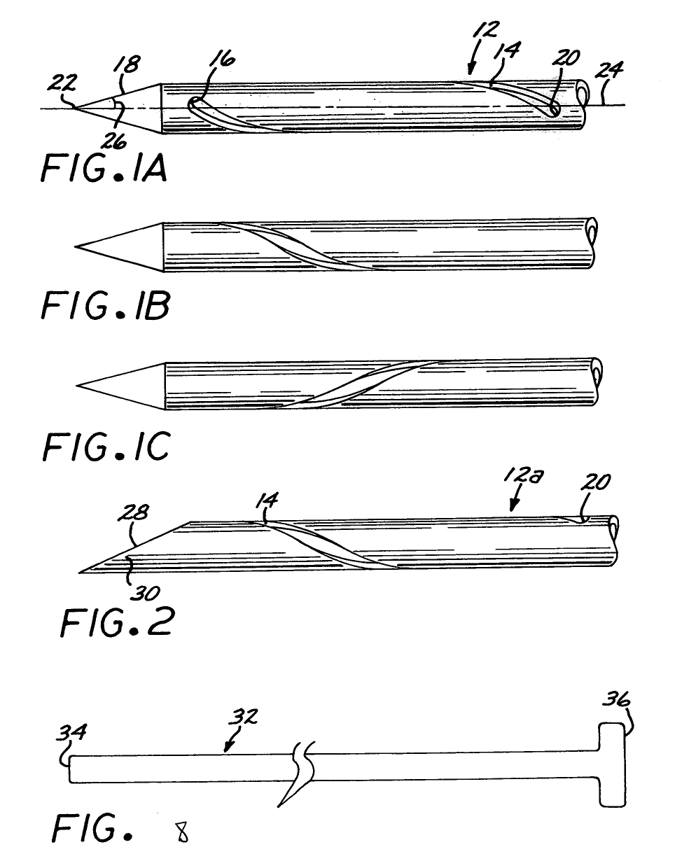

FIG. 2 illustrates an alternative embodiment 12a of the needle of the present invention wherein a beveled tip 28 is formed on the distal end of the needle. The tip is closed off and the bevel defines an angle 30 of approximately 22°. Other embodiments of this tip angle are envisioned to enhance tissue penetration.

The dimensions of the needle of the present invention are dependent upon the specific application for which the needle is intended. For use in a human knee, an 18 22 gauge stainless steel hypodermic needle approximately 2 3.5 inches in length is preferred. The proximal end of the needle may be fitted with a standard luer lock for use with a standard syringe.

The slot 14 may be formed in the needle using any of various standard manufacturing methods. A preferred method calls for the use of laser cutting or EDM. Slash grinding has also been successfully employed. The distal end of beveled needle may closed off by a weld while a closed of conical end may be formed by pressure rolling or swaging the tip closed.

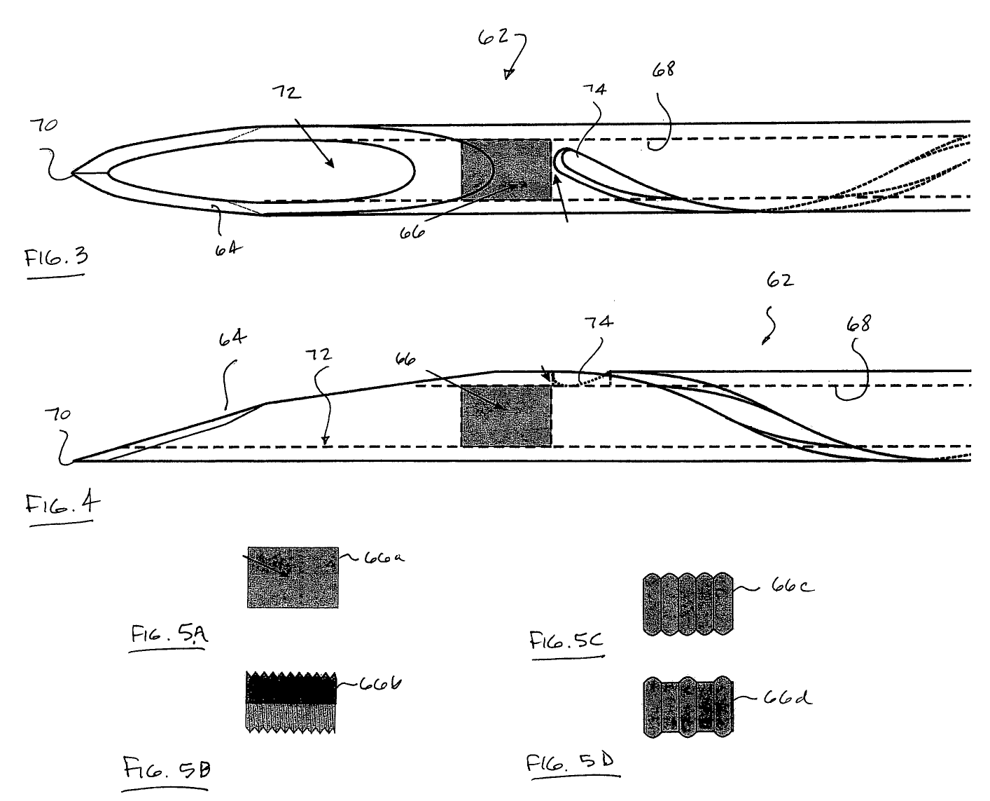

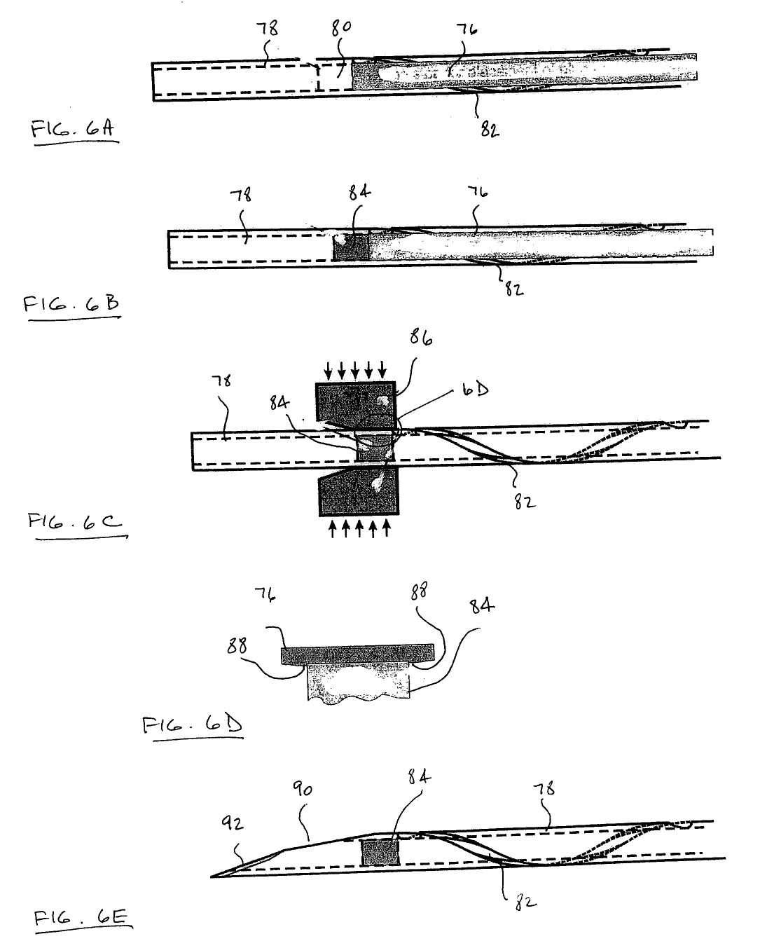

FIGS. 3 and 4 illustrate a preferred alternative embodiment of the present invention wherein the distal end of needle 62 is configured with a hollow bevel 64 in combination with plug 66 disposed within the needle lumen 68. The distal end of the hollow bevel terminates in a sharp tip 70 to facilitate the penetration of tissue while the hollow section 72 proximal thereto serves to reduce drag and friction during further insertion. The plug is preferably positioned just proximal to the proximal end of the bevel and just distal to the distal end of the slot 74. The plug may be welded or preferably swaged in place. In the event welding is relied upon to fix the plug in place within the needle lumen, a slight overlap between the proximal end of the plug and the distal end of the slot is desirable as the laser used to cut the slot may be relied upon to simultaneously weld a portion of the plug to the wall of the needle. In the event the plug is to be swaged in place a number of different surface geometries for the plug may be relied upon to enhance fixation and preclude slippage or dislodgement of the plug in the finished device. As an alternative to the smooth surface of the plug 66a shown in FIG. 5A, the plug 66b may have a threaded outer surface as shown in FIG. 5B, a multiple ribbed surface as shown in FIG. 5C or a three ribbed surface as shown in FIG. 5D. Each of the alternative plug configurations has a round cross-section and a diameter selected to provide a tight fight suitable for swage fixation. Additionally, the plug is to be formed of a material that is harder than that of the needle so as to allow the needle wall to deform around the plug and into any surface features it may have. The length of the plug is selected to be the minimum necessary to ensure positive fixation in light of the needle and plug material combination, the swaging force that is employed and the surface configuration of the plug.

FIGS. 6A-E illustrate a preferred method of manufacturing a needle in accordance with the present invention. As is shown in FIG. 6A, a rod stop is 76 is initially inserted into a hollow tube 78 such that its distal end is positioned immediately adjacent to the target plug placement site 80. A slot 82, preferably laser cut, may or may not have already been formed in the hollow tube. The plug 84 is subsequently inserted into the distal end of the tubing so as to engage the distal end of the rod stop as is shown in FIG. 6B. The rod stop is removed and a swaging device 86 is brought to bear on the exterior surface of the hollow tubing about the placement site (FIG. 6C). A sufficient amount of force is exerted so as to deform 88 the wall of the tubing 76 about the distal and proximal ends of the plug 84 as is visible in FIG. 6D. The stainless steel alloys of the plug and tubing are respectively selected such that the tubing is subject to deformation before the plug begins to deform. After the swaging operation is completed, a bevel 90 and preferably a double bevel 90, 92 is ground at an angle of about 8° and 16° so as to terminate at its proximal end at point just distal to the distal end of the plug and thereby ensure that the plug is positioned wholly within the interior of the tubing. In the event the slot had not initially been formed in the tubing, such slot may be formed at the end of the manufacturing process. The tip of the needle may optionally be subjected to silicone treatment to further reduce friction during penetration and advancement through tissue.

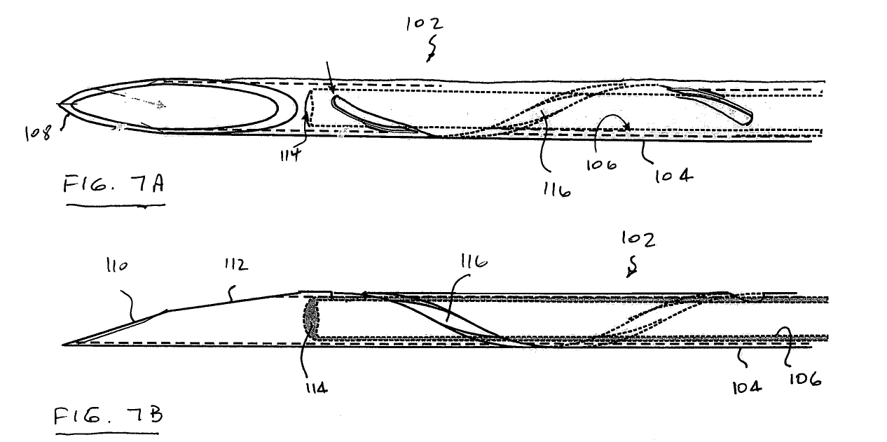

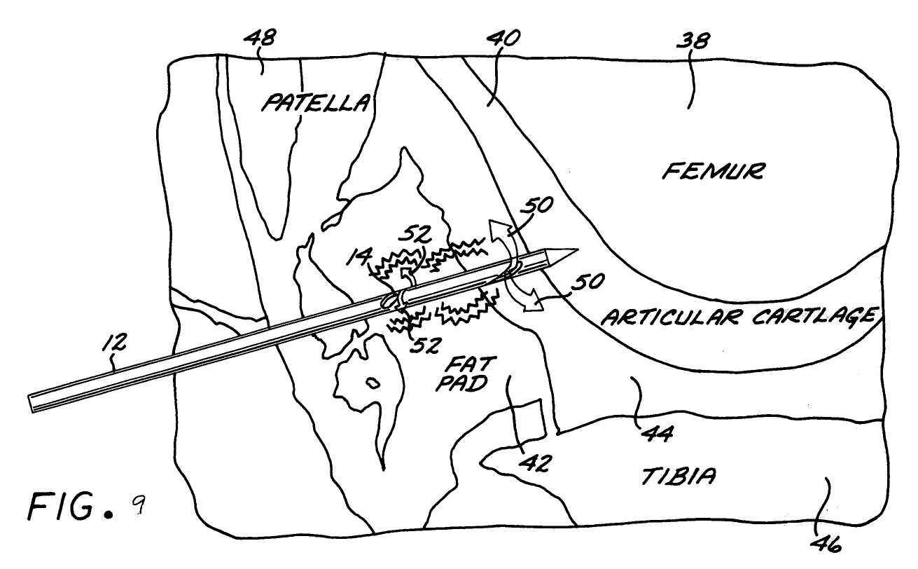

FIGS. 7A and 7B illustrate a most preferred needle configuration 102, wherein a hollow outer tube 104 is concentrically disposed about a hollow inner tube 106. The distal end of the outer tube is sharpened 108 and has a double bevel 110, 112 formed therein. The distal end 114 of the inner tube is closed off to effectively serve as a plug in the outer tube. The plug is positioned proximal of the proximal end of the bevel and distal of the distal end of the helical slot 116. The inner diameter of the outer tube and the outer diameter of the inner tube are selected to provide a very close fit there between. The helical slot 116 extends through both tube walls and is preferably dimensioned such that it is about ⅜″ in length, extends 360° around the needle and has a width corresponding to approximately ¼ to ⅓ of the needle's OD.

A preferred method of constructing the embodiment illustrated in FIGS. 7A and 7B first requires the selection of appropriate tubing sizes such that the inner diameter of the outer tube closely matches the outer diameter of the inner tube. A preferred example comprises “extra thin wall” 21 gage hypodermic tubing having an OD of 0.032″ in combination with “extra thin wall” 19 gage hypodermic tubing having an ID of 0.035″. Alternatively, 20 and 22 gage tubing provides a suitable combination. One end of the smaller tube is then closed off either by crimping or swaging and excess flash is cut off. The opposite end may then be flared or slitted after which the inner tube is inserted into the outer tube. A flared end limits the depth to which the smaller tube can be inserted into the larger tubing. A slitted configuration defines flaps that can bent outwardly 180° to engage the outer surface of the inner tube to similarly limit its insertion depth into the outer tube. Alternatively, the flaps are bent over after its insertion into the outer tube so as to engage and capture the outer tube. Once the two tubes are concentrically positioning, the helical slot is simultaneously laser cut into both tubes wherein the two tubes may become welded to one another to provide additional fixation causing the two needle tubes to function as a single tube or needle. The tip of the needle, and more specifically, the outer tube is ground so as to impart a double bevel and the appropriate sharpness to the distal tip. Optionally, the needle or just the needle tip may be subjected to a silicone treatment to further reduce insertion force and aid in skin penetration. Finally, a standard Luer lock hub assembly is fitted to the proximal end of the needle. The assembly is then appropriately cleaned, inspected, packaged and sterilized.

FIG. 8 illustrates a trochar 32 that may advantageously be used in combination with the needles illustrated in the Figures while the needle is being advanced through tissue into the joint. The outer diameter of the trochar is slightly less than the inner diameter of the needle while its distal end 34 may have a flat or rounded configuration. Its length must exceed the length of the needle and may have a manipulator element 36 fitted to its proximal to enable to exert a distally directed force thereon and to facilitate the retraction of the trochar after the needle has been placed.

FIG. 9 is a cross sectional view of a human knee showing the femur 38, the articular cartilage 40 that lines the femur, the fat pad 42 and the intraarticular space 44 situated therebetween. The tibia 46 and patella 48 are also visible. In use, the needle 12 is inserted into the fat pad just below the patella (anteromedial portal) and advanced therethrough until it impacts the bony wall of the intercondylar notch or the articular cartilage. A trochar may be used to aid in advancing the needle into place and to keep soft tissue from entering the lumen of the needle through the slot. The presence of the trochar significantly increases the needle's lateral stiffness and may be relied upon to enable a pushing force to be exerted directly on the interior surface of the distal tip rather than being transferred thereto across the slotted section. The use of the conical tip configuration as shown in FIGS. 1A-C rather than a beveled tip configuration as shown in FIG. 2 enhances the tracking of the needle to thereby more accurately follow the path intended by the clinician. A needle with a hollow bevel configuration as is illustrated in FIGS. 3, 4, 7A and 7B reduces the force necessary to advance the needle through tissue to thereby minimize trauma to the patient. The fact that the distal end of the needle is closed off in either configuration prevents the detachment of tissue through which it is being advanced by a cookie cutter effect and thereby prevents the subsequent injection of such tissue into the intraarticular space. The rounded shape of the proximal and distal ends of the slot 16, 20 similarly prevent the detachment of tissue as the needle is advanced therethrough. Once the needle is in place, the trochar is removed and a syringe is attached to the proximal end of the needle to facilitate the injection of viscous solutions or medication for example hyaluronan. The helical configuration of slot 14 guarantees that a direct flowpath 50 to the intraarticular space is established while the substantial backpressure created by the fat pad tissue density that surrounds the proximal portion of the slot prevents the escape of any substantial amount into the fat tissue (52). Similarly, back pressure of articular cartilage, ligamentous and/or connective tissue that surrounds the distal portion of the slot prevents the escape of any substantial amounts of fluid into these tissues.

While a particular form of the invention has been illustrated and described, it will also be apparent to those skilled in the art that various modifications can be made without departing from the spirit and scope of the invention. More particularly, the dimensions of the needle, including the width, length and positioning of the slot may be varied to accommodate different sized patients and different joints such as the shoulder or hip. The tip may be configured with different bevel angles, may be closed off using any of variety of plugs or seals and methods for attaching or forming such plugs or seals. Additionally, the needle can be used for injecting medicines such as those based on polymeric solutions for slow release or local release. Accordingly, it is not intended that the invention be limited except by the appended claims.

1. A device for injecting fluid, comprising a hollow needle with a lateral wall having a helical slot formed there through, a distal end defining a bevel and plug disposed within said needle proximal to said bevel and distal to said slot.

2. The device of claim 1, wherein needle comprises an outer tube concentrically disposed about an inner tube.

3. The device of claim 2, wherein said plug comprises a closed off distal end of said inner tube.

4. The device of claim 2, wherein said bevel is formed in said outer tube.

5. The device of claim 1, wherein said plug is swaged in place within said hollow needle.

6. The device of claim 5, wherein said plug has a smooth exterior surface.

7. The device of claim 5, wherein said plug has a threaded exterior surface.

8. The device of claim 5, wherein said plug has a ribbed exterior surface.

9. The device of claim 1, wherein said plug is welded in place.

10. The device of claim 1, further comprising a removable trochar configured to transfer a pushing force from the proximal end of said needle to said distal end to thereby isolate said lateral wall having said slot formed therein from such force.

11. A device for injecting fluid, comprising a needle having a lumen formed therein, a helical slot formed in said needle so as to set said lumen into fluid communication with the needle's exterior and a distal tip defining a hollow bevel wherein said lumen is closed off so as to isolate said distal tip from said lumen.

12. The device of claim 11, wherein said needle is double walled.

13. The device of claim 11, wherein said lumen is closed off at a point proximal to the proximal end of said bevel and distal to the distal end of said helical slot.

14. A method of forming a device for injecting fluid, comprising:
providing a first tube having an outer diameter;
providing a second tube having an inner diameter that is greater than said outer diameter of said first tube;
closing off the distal end of said first tube;
inserting said first tube into said second tube;
forming a helical slot that extends through both tubes; and
forming a bevel in the distal end of said outer tube.

15. The method of claim 14, wherein said closed off distal end of said first tube is positioned distal to the distal end of said helical slot and proximal to the proximal end of said bevel.

16. The method of claim 14, wherein said distal end of said first tube is closed off by swaging.

17. The method of claim 14, wherein said slot is formed by laser cutting.

18. The method of claim 14, wherein the proximal end of said first tube is flared prior to its insertion into said second tube.

19. The method of claim 14, wherein the proximal end of said first tube is slitted to form tabs and said tabs are bent over to capture the proximal end of said second tube.
